(19) United States
(12) Patent Application Publication
(10) Pub. No.: US 2005/0002964 A1
(43) Pub. Date: Jan. 6, 2005

(54) Adhesion inhibition of fungi

(76) Inventors: Dirk Bockmuhl, Wuppertal (DE); Roland Breves, Mettmann (DE); Mirko Weide, Dusseldorf (DE); Stefan Stumpe, Dusseldorf (DE); Michael Heinzel, Bonn (DE)

Correspondence Address: WOODCOCK WASHBURN LLP, ONE LIBERTY PLACE, 46TH FLOOR, PHILADELPHIA, PA, 19103, US

(21) Appl. No.: 10/871,124

(22) Filed: Jun. 18, 2004

Related U.S. Application Data
(63) Continuation of application No. PCT/EP02/14293, filed on Dec. 16, 2002.

(30) Foreign Application Priority Data
Dec. 18, 2001 (DE) DE 101 62 142.6

(51) Int. Cl. A61K 35/80; A61K 31/045; A61K 31/015

(52) U.S. Cl. 424/195.17; 424/539; 514/570; 514/738; 514/763

(57) ABSTRACT

The invention relates to the use of substances that influence fungal dimorphism for reducing the adhesion of fungi to surfaces. The invention also relates to washing and/or cleaning agents, textile treating agents and cleaning agents for mouth, teeth or dentures and to the use of said substances in said agents. The inventive substances are selected from propolis extracts, plant extracts, cinnamic acid and the derivatives thereof, monoterpenes, sesquiterpenes and diterpenes and the derivatives thereof, especially fucoidine, anethol, geraniol, farnesol or farnesol acid.

### CROSS REFERENCE TO RELATED APPLICATIONS

This application is a continuation of PCT/EP02/14293 filed Dec. 16, 2002, which claims the benefit of German Application No. DE 101 62 142.6, filed Dec. 18, 2001, the complete disclosures of which are hereby incorporated by reference in their entirety.

### FIELD OF THE INVENTION

The invention relates to the use of substances that influence the dimorphism of fungi so as to reduce the adhesion of fungi to surfaces and to detergent and/or cleaning agents, textile treating agents and oral, dental and dental prosthesis hygiene agents containing these substances.

Increasingly commonly, sensitive textiles, such as for example silk or microfibers, are used to produce articles of clothing which can only be washed at 30 or 40° C. Fungi, such as for example the human pathogen Candida albicans, are not killed by this. In particular, after a fungal infection, reinfection can occur because of such unkilled fungi adhering to articles of clothing.

Furthermore, wearers of dentures frequently contract oral candidiasis (thrush). Fungal cells adhering to the surface of the prosthesis can on contact colonize the mucous membranes, which have often previously been damaged because of pressure points.

In order to prevent reinfection by fungi adhering to the clothing or to plastic surfaces, antimicrobial substances were previously used, which either inhibit the growth of the fungi (fungistats) or kill them (fungicides). Non-selective antimicrobial substances, which act both against bacteria and also against fungi, are commonly used for this. The disadvantage with this is that such biocides or biostatic agents used for example in detergent and cleaning agents contaminate the waste water and thus also affect the function of the microbial purification stages in the water treatment plants. Moreover, the selection pressure on the microorganisms for the development of resistance is greatly increased, so that after some time new antimicrobial substances which are active against these microorganisms that have become resistant must be found.

### SUMMARY OF THE INVENTION

The purpose of the invention is therefore specifically to remove fungi from surfaces, without contaminating these surfaces or the waste water with fungicidal and/or fungistatic active substances.

The invention is based on the discovery that the adhesive behavior of fungi on surfaces can be regulated by influencing the dimorphism of fungi.

This purpose is achieved by the use of substances which influence the dimorphism of fungi, so as to reduce the adhesion of fungi onto surfaces.

According to the invention, substances which influence the dimorphism of fungi are used to reduce the adhesion of fungi onto surfaces. Here, dimorphism is understood to mean in general the possibility of the transition between two different vegetative cell forms, preferably between a filamentous and a unicellular form (in particular between a yeast form and a hyphal form). Advantageously, the unicellular forms adhere to fibers, textiles or plastic surfaces markedly worse than the filamentous forms. In the process, the growth of the cell is not inhibited, nor is it killed, rather the transformation of the fungal cell into another cell form is suppressed. The selection pressure for the development of resistance is therefore low.

In the meaning according to the invention, influencing should be understood to mean both the inhibition of the transition from one vegetative cell form into another and also the preferment or preferred formation of one particular cell form as opposed to another.

Reduction of adhesion should be understood to mean a significant reduction in the number of adhering fungal cells. Ideally, however, the adhesion is completely prevented. Preferably, the adhesion of fungal cells is decreased or essentially completely prevented.

A further advantage of the invention is that these substances are already active at low final concentrations compared to fungicides or fungistatic agents and thus side-effects are unlikely to be observed.

Furthermore, as a result of the decreased contact of the human body with the fungal cells, the reduction of the adhesion can also lead to a reduction in the allergy-triggering potential.

### DETAILED DESCRIPTION OF ILLUSTRATIVE EMBODIMENTS

According to a further particular embodiment, substances which inhibit the formation of hyphae are used to reduce the adhesion of fungi to surfaces.

An advantage of the use of substances which inhibit the formation of hyphae is that in these fungi no growth inhibition, but rather for example a transformation into the mostly pathogenic hyphal form (also called the filamentous or mycelial form) is prevented, or only still takes place to a slight extent and the cells thus remain in the less pathogenic yeast form. It has now been found that, in solutions which contain these substances (for example farnesol), the fungal cells adhere to surfaces scarcely or not at all.

According to a particular embodiment, the substances which influence the dimorphism of fungi, and especially preferably inhibit the formation of hyphae, are selected from propolis extracts, plant extracts, monoterpenes, sequiterpenes and/or diterpenes and derivatives thereof.

Acyclic, monocyclic and/or bicyclic mono-, sesqui- and/or diterpenes, and those with higher numbers of rings, can be used. Especially preferable are acylic terpenes. The derivatives of monoterpenes, sesqui-terpenes and diterpenes should be understood to mean for example alcohols, such as for example farnesol and ethers thereof, acids, such as for example forensic acid, and esters thereof and mono-, sesqui- or diterpenes bearing other functional groups. Here, both the trans and also the cis isomers are suitable.

Also among these is α-farnesene (3,7,11-trimethyl-1,3,6,10-dodecatetraene) and β-farnesene (7,11-di-methyl-3-methylene- 1,6,10-dodecatriene) and nerolidol (3,7,11-trimethyl-1,6,10-dodecatrien-3-ol) and also bisabolene, sesquiphellandrene, zingiberene, cadinene, caryophyllene (in particular α-caryophyllene (humulene) and β- caryophyllene), aryl-tumerone, tumerone, xanth-orrhizol, vulgarene and β-selinene. As monoterpenes, for example α- or βocimene, linalool, linalyl acetate, carene, terpineols, nerol, nerolic acid, geraniol, geranic acid, α- or β-phellandrene and/or thujone, in particular geraniol, linalool and/or thujone are especially suitable. As an example of the diterpenes, geranylgeraniol (3,7,11,15-tetramethyl-2,6,10,14-hexa-decatetraen- 1-ol) and isomers and derivatives thereof may be mentioned here. Plant extracts which contain mono-, sesqui- or diterpenes (for example geranium oil, rose oil, orange blossom oil, lavender oil, jasmine oil, basil oil, citronella oil, cypress oil, cedar leaf oil, coriander oil, rosewood oil, pimento oil, ginger oil or clove oil) can also preferably be used.

Cinnamic acid, in particular trans-cinnamic acid, and compounds derived therefrom with a double bond conjugated with the aromatic system can likewise be used as substances which influence the dimorphism of fungi and in particular inhibit the formation of hyphae. For example, these should be understood to include anethole (1-methoxy-4-(1-propenyl)benzene and other 3-phenylacrylic acid derivatives (or esters thereof). Plant extracts containing these substances, in particular aniseed oil and/or cinnamon oil, can likewise be used.

According to an especially preferred embodiment, the substances are selected from seaweed extracts, propolis extracts, fucoidin, farnesol, forensic acid, cinnamic acid, anethole, geraniol or other substances which influence the dimorphism of fungi. Especially preferred are propolis extracts and farnesol. Fucoidin, also known under the names fucosidan or fucoidan, is a polysaccharide from brown seaweeds (Fucus vesiculosa, bladderwrack), which consists mainly of sulfated L-fucose with 1,2-α-glycoside linkages. It has advantageously been found that the formation of hyphae by Candida albicans is significantly reduced, while at the same time the cell growth is not affected.

Propolis is a resinous mass with a melting point between ca. 50 and 70° C., which is collected by bees and is used in the beehive as a coating for the walls and to reinforce the honeycomb. It is also known as filling wax, bee cement or bee resin. Propolis is thus mainly obtained by removing (e.g. scraping off) this coating from the honeycombs and walls of the beehive or by means of so-called propolis gratings which are laid over the frames of the beehive and coated with propolis by the bees.

Plant extracts, especially seaweed extracts, and propolis extracts, can be extracted therefrom in ways know to the skilled person, with water, and polar or nonpolar organic solvents and mixtures thereof. Extracts which can be obtained by extraction with ethanol or water/ethanol mixtures, and in particular propolis extracts obtained in such a manner, are particularly preferred.

According to a particular embodiment, the substances which influence the dimorphism of fungi are used at final concentrations which do not have fungicidal (fungus-killing) or fungistatic (fungal growth-inhibiting) action. A particular advantage of this embodiment is that the risk of development of resistance to the substances used is relatively low, since the fungi are neither killed, nor is their growth inhibited. The concentrations at which there is still no inhibition of growth, and the minimum inhibitory concentrations themselves can be simply determined in the manner known to the skilled person.

According to a further particular embodiment, the substances which influence the dimorphism of fungi are contained in an amount of 0.000001 to 3 weight %. A particular advantage of this embodiment is that only low concentrations of these substances have to be present for the adhesion of the fungi to surfaces to be reduced or essentially completely prevented.

Preferably the substances are contained in an amount of 0.00001 to 1 weight % and in particular 0.0001 to 0.5 weight %. Particularly preferred are ranges between 0.0001 and 0.1 weight %. Specifically in the case of fucoidan, amounts between 0.0001 and 0.5 weight % are particularly preferred.

The concentrations which lead to the desired result in the final product are markedly lower than those stated, since for many products dilutions have to be taken into account. For example, for detergents a dilution factor (ratio of detergent concentrate:water) of 1:20 to 1:200 must be allowed for. Often the dilution ratio for detergents is between 1:60 and 1:100, for example 1:80.

For farnesol, for example concentrations from 0.001 to 1.5 weight %, in particular from 0.01 to 0.8 weight % are suitable.

According to a further particular embodiment, the adhesion of fungi pathogenic to humans is reduced by the use of substances which influence the dimorphism of fungi. These include for example the species pathogenic to humans of the classes Ascomycota, Basidiomycota, Deuteromycota and Zygomycota, in particular the forms of Candida pathogenic to humans.

The Candida species pathogenic to humans also colonize skin and mucous membranes in healthy persons. However, on intense multiplication of the fungal cells, e.g. after impairment of the mucosal bacterial flora by antibiotics, they cause local inflammations, which are also described as thrush. These occur in particular in the oral and genital region (so-called oral or vaginal thrush). Also known are skin and nappy thrush. The mucous membrane is reddened, lesions appear, and a white furring and itching occur.

According to a further especially preferred embodiment, adhesion of fungi of the Candida species (abbreviated below to C.) such as for example: C. aaseri, C. actis-condensi, C. acutus, C. agrestis, C. albicans, C. amapae, C. anatomiae, C. ancudensis, C. antarctica, C. antillancae, C. apicola, C. apis, C. aquaetextoris, C. aquatica, C. atlantica, C. atmosphaerica, C. auringiensis, C. azyma, C. beechii, C. benhamii, C. bertae, C. berthetii, C. blankii, C. boidinii, C. boleticola, C. bombi, C. bondarzewiae, C. brumptii, C. buffonii, C. buinensis, C. cacaoi, C. canterellii, C. capsuligena, C. cariosilignicola, C. caseinolytica, C. castellii, C. catenulata, C. chalmersi, C. chilensis, C. chiropterorum, C. ciferii, C. claussenii, C. coipomensis, C. colliculosa, C. conglobata, C. curiosa, C. cylindracea, C. dendrica, C. dendronema, C. deserticola, C. diddensiae, C. diffluens, C. diversa, C. drymisii, C. dubliniensis, C. edax, C. entomophila, C. eremophila, C. ergatensis, C. ernobii, C. etchellsii, C. etchellsii, C. ethanolica, C. ethanothermophilum, C. evantina, C. fabianii, C. famata, C. fennica, C.flareri, C fluviotilis, C. fragariorum, C. fragi, C. fragicola, C. freyschussii, C. friedrichii, C. fructus, C. fusiformata, C. geochares, C. glabrata, C. glaebosa, C. graminis, C. gropengiesseri, C. guilliermondii, C. haemulonii, C. hellenica, C. heveanensis, C. holmii, C. homilentoma, C. humicola, C. humilis, C. iberica, C. incommunis, C. inconspicua, C. ingens, C. insectalens, C. insectamans, C. insectorum, C. intermedia, C. ishiwadae, C. japonica, C. javanica, C. karawaiewii, C. kefyr, C. kruisii, C. krusei, C. krusoides, C. lactiscondensi, C. lambica, C. laureliae, C. lipolytica, C. llanquihuensis, C. lodderae, C. lusitaniae, C. magnoliae, C. malicola, C. maltosa, C. maris, C. maritima, C. melibiosica, C. melinii, C. membranaefaciens, C. mesenterica, C. methanosorbosa, C. miller, C. mogii, C. molischiana, C. monosa, C. montana, C. mucilaginosa, C. multisgemmis, C. musae, C. muscorum, C. mycoderma, C. naeodendra, C. nakasei, C. nemodendra, C. nitratophila, C. norvegensis, C. novakii, C. oleophila, C. oregonensis, C. palmyrana, C. paludigena, C. parapsilosis, C. pararugosa, C. pelliculosa, C. peltata, C. periphelosum, C. petro-huensis, C. pignaliae, C. pintolopesii, C. pinus, C. placentae, C. polymorpha, C. populi, C pseudo-tropicalis, C. psychrophila, C. pulcherrima, C. punica, C. quercitrusa, C. quercuum, C. railenensis, C. ralunensis, C. reukaufii, C. rhagii, C. rugo-pelliculosa, C. rugosa, C. saitoana, C. sake, C. salmanticensis, C. santamariae, C. santjacobensis, C. savonica, C. schatavii, C. sequanensis, C. shehatae, C. silvae, C. silvanorum, C. silvicultrix, C. solani, C. sonorensis, C. sophiae-reginae, C sorboxylosa, C. spandovensis, C. sphaerica, C. stellata, C. stellatoidea, C. succiphila, C. sydowiorum, C. tanzawaensis, C. tenuis, C. tepae, C. terebra, C. torresii, C. tropicalis, C. tsuchiyae, C. tsukubaensis, C. utilis, C. valdiviana, C. valida, C. vanderwaltii, C. vartiovaarai, C. versatilis, C. vini, C. viswanathii, C wickerhainii, C. xestobii and C. zeylanoides is reduced.

In particular, the adhesion of the medically important forms of Candida, for example of: C. albicans, C. boidinii, C. catenulata, C. ciferii, C. dubliniensis, C. glabrata, C. guilliermondii, C. haemulonii, C. kefyr, C. krusei, C. lipolytica, C. lusitaniae, C. norvegensis, C. parapsilosis, C. pulcherrima, C. rugosa, C. tropicalis, C. utilis and C. viswanathii is reduced by the use according to the invention. Especially preferred are C. albicans, C. stelladoidea, C. tropicalis, C. glabrata and C. parapsilosis. The mycelial form of Candida is regarded as the form of the fungus pathogenic in humans. A reduction of the adhesion of Candida for example to textiles or plastics reduces the risk of reinfection, without increasing the development of resistance.

According to a particular embodiment, the adhesion of fungi to surfaces which often come into contact with the human body is reduced or essentially completely prevented. Here, abiotic surfaces in particular are meant. Hence in the sense of the invention, this should not be understood to include human tissue.

With inadequate cleaning of these surfaces, reinfection of already affected parts of the body or further new infections can occur as a result of the adhesion of fungi.

According to a quite especially preferred embodiment, the adhesion of fungi to surfaces such as textiles, ceramics, metals and/or plastics is reduced. These are in particular laundry, prostheses or dentures. Fungal infections of mucous membranes, especially in the oral and genital area, can be simply and successfully treated with antimycotic agents. Here, however, it is very important that the surfaces contaminated with fungal cells, for example underwear, are cleared of these. In the case of sensitive textiles, such as for example silk or microfibers and synthetic fabrics, this cannot be effected by means of a higher wash temperature without the material being damaged. The use of strong bleach-containing all-purpose washing powders is also not to be recommended because of possible damage to the materials.

The reduction of the adhesion to textiles or plastic surfaces very often prevents reinfection of the body areas already affected. The reduction of the adhesion to ceramics, plastics or metals, in particular to prostheses or dentures, decreases the risk of infection or reinfection, without contaminating the skin, the mucous membranes or the waste water with substances with fungicidal or fungistatic activity. Likewise, catheters and other medical devices and/or prostheses made of plastic or metal can be freed from fungal adhesion by the use of such substances for example in douches or cleaning agents.

According to a further particular embodiment, the substances which influence the dimorphism of fungi are added to detergent and/or cleaning agents or to oral hygiene or denture cleaning products. In particular, the modem textile fibers which cannot be washed with all-purpose detergents or at high temperatures cannot be completely freed from fungal adhesions by normal mild detergents or wash temperatures at 30 or 40° C. An advantage of the use of such additives which influence the dimorphism of fungi in detergent and cleaning agents is that in spite of the low waste water contamination and the low risk of development of resistance, articles of clothing can be freed from the adhesion of fungi.

Through the use of such substances in oral, dental and/or dental prosthesis hygiene products, dental prostheses, in particular dentures, can be cleared of fungal adhesion simply and without contamination of the treated surfaces with strongly fungicidal, and possibly under some circumstances toxic, substances. Propolis and/or seaweed extracts are particularly suitable for oral, dental and/or dental prosthesis hygiene.

Also an object of the invention are detergent and/or cleaning agents containing 0.000001 to 3 weight % of substances which influence the dimorphism of fungi. Particularly preferred are concentrations of 0.00001 to 1.0 weight % and in particular 0.0001 to 0.5 weight %. Quite especially preferably, the detergent and cleaning agents contain 0.0001 to 0.05 weight % of these substances.

Especially suitable are substances which inhibit the formation of hyphae. Advantageously, in the unicellular yeast form the fungal cells adhere markedly less to surfaces such as for example textiles or plastics. Such detergent and cleaning agents can without contamination of the waste water contain relatively small amounts of substances influencing the dimorphism of fungi. Since they are used in concentrated form and diluted to the appropriate active concentrations in the wash liquor, the active substances must be used in correspondingly higher concentration. Dilutions of the detergent and cleaning agents with water of between 1:40 and 1:200 are usual.

Such substances can according to the invention also be added to cleaning agents which for the cleaning of hard surfaces, such as for example floors, tiles, floor tiles, plastics and other hard surfaces in the home or in medical practice.

In the context of the invention, detergent and cleaning agents are understood to mean in the widest sense surfactant-containing preparations in solid form (particles, powders and the like), semi-solid form (pastes and the like), liquid form (solutions, emulsions, suspensions, gels and the like) and gas-like form (aerosols and the like), which for the purposes of advantageous action during use contain one surfactant or several surfactants, usually in addition to other components, which are usual for the given use purpose. Examples of such surfactant-containing preparations are surfactant-containing detergent preparations, surfactant-containing cleaning agents for hard surfaces, or surfactant-containing brightener preparations, which can each be solid or liquid, but can also be in a form which includes solid and liquid components or part amounts of the components together.

The detergent and cleaning agents can contain normally contained ingredients, such as anionic, nonionic, cationic and amphoteric surfactants, inorganic and organic builders, special polymers (for example those with cobuilder properties), foam inhibitors, colorants and optionally additional aroma substances (perfumes), bleaching agents (such as for example peroxo bleaching agents and chlorine bleaching agents), bleach activators, bleach stabilizers, bleach catalysts, enzymes and graying inhibitors, without the ingredients being limited to these substance groups. Often, important ingredients of these preparations are also washing aids, which are understood to mean, by way of example and not in a limiting sense, optical brighteners, UV protection agents and so-called soil repellents, i.e. polymers which counteract repeat soiling of fibers. The individual substance groups are explained in more detail below.

In the event that the preparations are at least in part in the form of molded articles, they may also contain binding and disintegration aids.

As surfactants, anionic, nonionic, zwitterionic and cationic surfactants can be used.

As anionic surfactants, for example those of the sulfonate or sulfate type are used. Possible surfactants of the sulfonate type are preferably C₉₋₁₃ alkylbenzenesulfonates, olefin sulfonates, i.e. mixtures of alkene- and hydroxyalkane-sulfonates and disulfonates, such as are for example obtained from C₁₂₋₁₈ mono-olefins with a terminal or internal double bond by sulfonation with gaseous sulfur trioxide, followed by alkaline or acid hydrolysis of the sulfonation products. Also suitable are alkanesulfonates, which are obtained from C₁₂₋₁₈ alkanes for example by sulfochlorination or sulfoxidation followed by hydrolysis or neutralization. Likewise, the esters of 2-sulfo-fatty acids (ester sulfonates), e.g. the 2-sulfonated methyl esters of hydrogenated coconut, palm nut or tallow fatty acids are also suitable.

Further suitable anionic surfactants are sulfated fatty acid glycerin esters. Fatty acid glycerin esters are understood to mean the mono-, di- and triesters and mixtures thereof, such as are obtained in the preparation by esterification of a monoglycerin with 1 to 3 moles of fatty acid or in the trans-esterification of triglycerides with 0.3 to 2 moles of glycerin. Preferred sulfated fatty acid glycerin esters here are the sulfation products of saturated fatty acids with 6 to 22 carbon atoms, for example caproic acid, caprylic acid, capric acid, myristic acid, lauric acid, palmitic acid, stearic acid or behenic acid.

As alk(en)yl sulfates, the alkali metal and in particular the sodium salts of the sulfuric acid half esters of the C₁₂-C₁₈ fatty alcohols, for example from coconut fatty alcohol, tallow fatty alcohol, lauryl, myristyl, cetyl or stearyl alcohol or the C₁₀-C₂₀ oxo alcohols and such half-esters of secondary alcohols of these chain lengths. Also preferred are alk(en)yl sulfates of the said chain length, which contain a synthetic, straight-chain alkyl residue made from petrochemicals, which have an analogous degradation behavior to the equivalent compounds based on fat chemistry raw materials. In detergent and cleaning agents, the C₁₂-C₁₆ alkyl sulfates and C₁₂-C₁₅ alkyl sulfates and C₁₄-C₁₅ alkyl sulfates are preferred. 2,3-alkyl sulfates, which can for example be obtained in accordance with U.S. Pat. Nos. 3,234,258 or 5,075,041 and as commercial products of the Shell Oil Company under the name DAN®, are also suitable anionic surfactants.

The sulfuric acid mono esters of the straight-chain or branched C₇₋₂₁ alcohols ethoxylated with 1 to 6 moles of ethylene oxide, such as 2-methyl branched C₉ alcohols with on average 3.5 moles ethylene oxide (EO) or C₁₂₋₁₈ fatty alcohols with 1 to 4 EO, are also suitable. On account of their marked foaming behavior, they are used in detergent and cleaning agents only in relatively small amounts, for example in amounts of 1 to 5 weight %.

Further suitable anionic surfactants are also the salts of alkylsulfosuccinic acid, which are also described as sulfosuccinates or as sulfosuccinate esters, and are the monoesters and/or diesters of sulfosuccinic acid with alcohols, preferably fatty alcohols and in particular ethoxylated fatty alcohols. Preferred sulfosuccinates contain C₈₋₁₈ fatty alcohols or mixtures thereof. Especially preferred sulfosuccinates contain a fatty alcohol residue which is derived from ethoxylated fatty alcohols, which considered per se are nonionic surfactants (see below for description). Here, however, sulfosuccinates whose fatty alcohol residues are derived from ethoxylated fatty alcohols with a restricted homolog distribution are especially preferred. Likewise it is also possible to use alk(en)ylsuccinic acid with preferably 8 to 18 carbon atoms in the alk(en)yl chain or salts thereof.

Soaps in particular are possible as further anionic surfactants. Saturated fatty acid soaps, such as the salts of lauric acid, myristic acid, palmitic acid, stearic acid, hydrogenated erucic acid and behenic acid and in particular soap mixtures derived from natural fatty acids, e.g. coconut, palm nut or tallow fatty acids, derived soap mixtures are suitable.

The anionic surfactants including the soaps can be present in the form of their sodium, potassium or ammonium salts and as soluble salts of organic bases, such as mono-, di-or triethanolamine. The sodium or potassium salts, in particular the sodium salts, are preferred. The surfactants can also be used in the form of their magnesium salts.

In the context of the present invention, agents which contain 5 to 50 weight %, preferably 7.5 to 40 weight % and in particular 15 to 25 weight % of one or several anionic surfactant(s) are preferred.

As nonionic surfactants, alkoxylated, advantageously ethoxylated, in particular primary alcohols with preferably 8 to 18 C atoms and on average 1 to 12 moles of ethylene oxide (EO) per mole of alcohol, wherein the alcohol residue can be linear or preferably methyl-branched in the 2-position or can contain a mixture of linear and methyl-branched residues, such as are usually present in oxo alcohol residues, are preferably used. In particular, however, alcohol ethoxylates with linear residues from alcohols of natural origin with 12 to 18 carbon atoms, e.g. from coconut, palm, tallow fat or oleyl alcohol, and on average 2 to 8 EO per mole of alcohol, are preferred. The preferred ethoxylated alcohols for example include C₁₂₋₁₄ alcohols with 3 EO or 4 EO, C₉₋₁₁ alcohol with 7 EO, C₁₃₋₁₅ alcohols with 3 EO, 5 EO, 7 EO or 8 EO, C₁₂₋₁₈ alcohols with 3 EO, 5 EO or 7 EO and mixtures of these, and mixtures of C₁₂₋₁₄ alcohol with 3 EO and C₁₂₋₁₈ alcohol with 5 EO. The stated ethoxylation levels are statistical average values, which for a specific product can be a whole or a fractional number. Preferred alcohol ethoxylates have a restricted homolog distribution (narrow range ethoxylates, NRE). In addition to these nonionic surfactants, fatty acid alcohols with more than 12 EO can also be used. Examples of these are tallow fatty alcohol with 14 EO, 25 EO, 30 EO or 40 EO.

A further class of preferably used nonionic surfactants, which are used either as a nonionic surfactant alone or in combination with other nonionic surfactants, are alkoxylated, preferably ethoxylated or ethoxylated and propoxylated fatty acid alkyl esters, preferably with 1 to 4 carbon atoms in the alkyl chain, in particular fatty acid methyl esters.

A further class of nonionic surfactants which can be advantageously used are the alkylpolyglycosides (APG). Alkylpolyglycosides that can be used satisfy the general formula RO(G)_{z}, wherein R stands for a linear or branched, in particular methyl-branched in the 2-position, saturated or unsaturated, aliphatic residue with 8 to 22, preferably 12 to 18 C atoms and G is the symbol that stands for a glycose unit with 5 or 6 C atoms, preferably for glucose. The glycosylation level z here lies between 1.0 and 4.0, preferably between 1.0 and 2.0 and in particular between 1.1 and 1.4.

Linear alkylpolyglucosides, i.e. alkylpolyglycosides, wherein the polyglycosyl residue is a glucose residue and the alkyl residue is an n-alkyl residue are preferably used.

The surfactant-containing preparations according to the invention can preferably contain alkylpolyglycosides, APG contents of the preparations intended for washing, rinsing or cleaning purposes of over 0.2 weight %, based on the whole preparation, being preferred. Especially preferred surfactant-containing preparations contain APG in amounts from 0.2 to 10 weight %, preferably in amounts from 0.2 to 5 weight % and in particular in amounts from 0.5 to 3 weight %.

Nonionic surfactants of the amine oxide type, for example N-coconut-alkyl-N,N-dimethylamine oxide and N-tallow-alkyl-N,N-dihydroxyethylamine oxide and the fatty acid alkanolamides can be suitable. The amount of these nonionic surfactants is preferably not more than that of the ethoxylated fatty alcohols, in particular not more than half thereof.

Further suitable surfactants are polyhydroxy fatty acid amides of the formula (I), 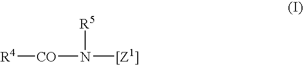
wherein R⁴CO stands for an aliphatic acyl residue with 6 to 22 carbon atoms, R⁵ for hydrogen, an alkyl or hydroxyalkyl residue with 1 to 4 carbon atoms and [Z¹] for a linear or branched polyhydroxyalkyl residue with 3 to 10 carbon atoms and 3 to 10 hydroxy groups. The polyhydroxy fatty acid amides are known substances, which can usually be obtained by reductive amination of a reducing sugar with ammonia, an alkylamine or an alkanolamine, followed by acylation with a fatty acid, a fatty acid alkyl ester or a fatty acid chloride.

The group of polyhydroxy fatty acid amides also includes compounds of the formula (II), 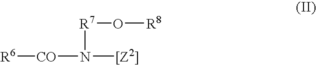
wherein R⁶ stands for a linear or branched alkyl or alkenyl residue with 7 to 12 carbon atoms, R⁷ for a linear, branched or cyclic alkyl residue or an aryl residue with 2 to 8 carbon atoms and R⁸ for a linear, branched or cyclic alkyl residue or an aryl residue or a hydroxyalkyl residue with 1 to 8 carbon atoms, C ₁₋₄ alkyl or phenyl residues being preferred, and [Z²] stands for a linear polyhydroxyalkyl residue, whose alkyl chain is substituted with at least two hydroxy groups, or alkoxylated, preferably ethoxylated or propoxylated derivatives of this residue.

[Z²] is preferably obtained by reductive amination of a reduced sugar, for example glucose, fructose, maltose, lactose, galactose, mannose or xylose. The N-alkoxy or N-aryloxy substituted compounds can then for example, as described in WO-A-95/07331, be converted into the desired polyhydroxy fatty acid amides by reaction with fatty acid methyl esters in the presence of an alkoxide as catalyst.

Further, it can be preferable, in addition to anionic and nonionic surfactants, also to use cationic surfactants.

Cationic surfactants can be mentioned in particular as textile-softening substances. Examples of cationic surfactants are in particular quaternary ammonium compounds, cationic polymers and emulsifiers.

Suitable examples are quaternary ammonium compounds of the formulae (III) and (IV) 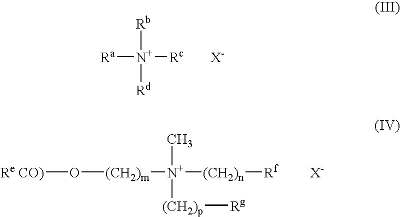
where, in (IV), R^{a} and R^{b} stand for an acyclic alkyl residue with 12 to 24 carbon atoms, R^{c} for a saturated C₁-C₄ alkyl or hydroxyalkyl residue, and R^{d} is either the same as R^{a}, R^{b} or R^{c} or stands for an aromatic residue. X⁻ stands either for a halide, methosulfate, methophosphate or phosphate ion and mixtures thereof. Examples of cationic compounds of the formula (III) are didecyldimethylammonium chloride, ditallowdimethyl-ammonium chloride or dihexadecylammonium chloride.

Compounds of the formula (IV) are so-called ester quats. Ester quats are characterized by outstanding biological degradability. Herein, R^{e} stands for an aliphatic acyl residue with 12 to 22 carbon atoms with 0, 1, 2 or 3 double bonds; R^{f} stands for H, OH or O(CO)R^{h}, R^{g} independently of R^{f} stands for H, OH or O(CO)Rⁱ, where R^{h} and Rⁱ mutually independently each stands for an aliphatic acyl residue with 12 to 22 carbon atoms with 0, 1, 2 or 3 double bonds. m, n and p can each mutually independently have the value 1, 2 or 3. X⁻ can be either a halide, methosulfate, methophosphate or phosphate ion or mixtures thereof. Compounds which contain the group O(CO)R^{h} for R^{f} and contain alkyl residues with 16 to 18 carbon atoms for R^{c} and R^{h} are preferred. Especially preferred are compounds wherein in addition R^{g} stands for OH. Examples of compounds of the formula (IV) are methyl N-(2-hydroxyethyl)-N,N-di(tallow acyloxyethyl)ammonium methosulfate, bis-(palmitoyl)-ethyl-hydroxyethyl-methyl-ammonium methosulfate or methyl N,N-bis(acyloxy-ethyl)-N-(2-hydroxyethyl)ammonium methosulfate. If quaternized compounds of the formula (IV) which possess unsaturated alkyl chains are used, the acyl groups whose corresponding fatty acids have an iodine number between 5 and 80, preferably between 10 and 60 and in particular between 15 and 45 and which have a cis/trans isomer ratio (in weight %) of greater than 30:70, preferably greater than 50:50 and in particular greater than 70:30 are preferred. Normal commercial examples are the methylhydroxylalkyldialkoyloxyalkylammonium methosulfates marketed by Stepan under the brand name Stepantex® or the products of Cognis known as Dehyquart® or the products of Goldschmidt-Witco known as Rewoquat®. Further preferred compounds are the diester quats of the formula (V), which are obtainable under the name Rewoquat® W 222 LM or CR 3099 and in addition to softness also provide stability and color fastness. 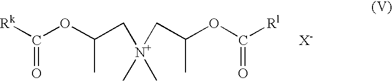
wherein R^{k} and R^{l} mutually independently each stand for an aliphatic acyl residue with 12 to 22 carbon atoms with 0, 1, 2 or 3 double bonds.

In addition to the quaternary compounds described above, other known compounds can also be used, such as for example quaternary imidazolinium compounds of the formula (VI), 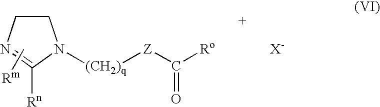
wherein R^{m} can stand for H or a saturated alkyl residue with 1 to 4 carbon atoms, Rⁿ and R^{o} each mutually independently for an aliphatic, saturated or unsaturated alkyl residue with 12 to 18 carbon atoms, Rⁿ alternatively also for O(CO)R^{p}, wherein R^{p} means an aliphatic, saturated or unsaturated alkyl residue with 12 to 18 carbon atoms, and Z means an NH group or oxygen and X⁻ is an anion. q can take whole number values between 1 and 4.

Further suitable quaternary compounds are described by the formula (VII), 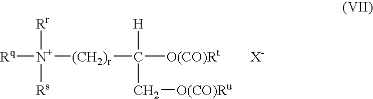
wherein R^{q} R^{r} and R^{s} mutually independently stand for a C₁₋₄ alkyl, alkenyl or hydroxyalkyl group, R^{t} and R^{u} each independently selected represents a C₈₋₂₈ alkyl group and r is a number between 0 and 5.

In addition to the compounds of the formulae III to VII, short-chain, water-soluble, quaternary ammonium compounds, such as trihydroxyethylmethylammonium methosulfate or the alkyltrimethylammonium chlorides, dialkyldimethylammonium chlorides and trialkylmethyl-ammonium chlorides, e.g. cetyltrimethylammonium chloride, stearyltrimethylammonium chloride, distearyl-dimethylammonium chloride, lauryldimethylammonium chloride, lauryldimethylbenzylammonium chloride and tricetylmethylammonium chloride, can also be used.

Protonated alkylamine compounds, which have a softening action, and the non-quaternized, protonated precursors of the cationic emulsifiers are also suitable.

Further cationic compounds usable according to the invention are the quatemized protein hydrolysates.

The suitable cationic polymers include the poly-quaternium polymers, such as in the CTFA Cosmetic Ingredient Dictionary (The Cosmetic, Toiletry and Fragrance, Inc., 1997), in particular the poly-quaternium-6, polyquaternium-7 and polyquaternium- 10 polymers also described as Merquats (Ucare Polymer IR 400; Amerchol), polquaternium-4 copolymers such as graft copolymers with a cellulose skeleton and quaternary ammonium groups which are linked via allyldimethylammonium chloride, cationic cellulose derivatives, such as cationic guar, such as guar hydroxypropyltriammonium chloride, and similar quaternized guar derivatives (e.g. Cosmedia Guar, manufacturer: Cognis GmbH), cationic quaternary sugar derivatives (cationic alkylpolyglucosides), e.g. the commercial product Glucquat® 100, according to the CTFA nomenclature a “lauryl methyl gluceth-10 hydroxypropyl dimonium chloride”, copolymers of PVP and dimethylaminomethacrylate, copolymers of vinylimidazole and vinylpyrrolidone, aminosilicone polymers and copolymers,

Also usable are polyquatemized polymers (e.g. Luviquat Care from BASF) and also chitin-based cationic biopolymers and derivatives thereof, for example the polymer obtainable under the trade name Chitosan® (manufacturer: Cognis).

Also suitable according to the invention are cationic silicone oils such as for example the commercially available products Q2-7224 (manufacturer: Dow Coming; a stabilized trimethylsilylamodimethicone), Dow Coming 929 Emulsion (containing a hydroxyl-amino modified silicone, which is also described as amodimethicone), SM-2059 (manufacturer: General Electric), SLM-55067 (manufacturer: Wacker) Abil®-Quat 3270 and 3272 (manufacturer: Goldschmidt-Rewo; diquatemary poly-dimethylsiloxane, Quatemium-80), and Siliconequat Rewoquat® SQ1 (Tegopren® 6922, manufacturer: Goldschmidt-Rewo).

Also usable are compounds of the formula (VIII) 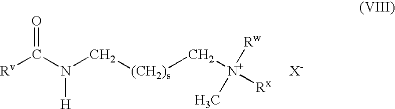
which can be alkylamidoamines in their non-quatemized form, or, as shown, their quatemized form. R^{V} can be an aliphatic acyl residue with 12 to 22 carbon atoms with 0, 1, 2 or 3 double bonds. s can take values between 0 and 5. R^{W} and R^{X} each mutually independently stand for H, C₁₋₄ alkyl or hydroxyalkyl. Preferred compounds are fatty acid amidoamines such as the stearylamidopropyldimethylamine obtainable under the name Tego Amid® S 18 or the 3-tallow-amidopropyl-trimethylammonium methosulfate obtainable under the name Stepantex® X 9124, which in addition to good conditioning action are also characterized by color transfer-inhibiting action and especially by their good biological degradability.

If cationic surfactants are used, they are preferably contained in the preparations in amounts from 0.01 to 10 weight %, in particular from 0.1 to 3.0 weight %.

The total surfactant content in the agents according to the invention can lie between 5 and 50 weight %, preferably between 10 and 35 weight %.

Apart from the surfactants, builders are the most important components of detergent and cleaning agents. The surfactant-containing preparations according to the invention can containing builders normally used in detergent and cleaning agents, i.e. in particular zeolites, silicates, carbonates, organic cobuilders and also—where there are no ecological prejudices against their use—the phosphates.

Suitable crystalline, laminar sodium silicates have the general formula NaMSiₓO₂ₓ₊₁.H₂O, wherein M means sodium or hydrogen, x is a number from 1.9 to 4 and y a number from 0 to 20 and preferred values for x are 2, 3 or 4. Such crystalline laminar silicates are for example described in the European patent application EP-A-0 164,514. Preferred crystalline laminar silicates of the stated formula are those wherein M stands for sodium and x takes the values 2 or 3. In particular, both β- and also δ-sodium disilicates Na₂Si₂O_{5.y}H₂O are preferred, where β-sodium disilicate for example can be obtained by the procedure which is described in the international patent application WO-A-91/08171.

Also usable are amorphous sodium silicates with an Na₂O:SiO₂ modulus from 1:2 to 1:3.3, preferably from 1:2 to 1:2.8 and in particular from 1:2 to 1:2.6, which are solution-retarded and have secondary detergent properties. Here, the solution retardation compared to conventional amorphous sodium silicates can be induced in various ways, for example by surface treatment, compounding, compacting/compression or by superdrying. So-called X-ray-amorphous silicates, which likewise display solution retardation compared to the conventional waterglasses, are for example described in the German patent application DE-A 44 00 024. The products have microcrystalline regions 10 to a few hundred nm in size, values up to max. 50 nm and in particular up to max. 20 nm being preferred. Especially preferred are compressed/compacted amorphous silicates, compounded amorphous silicates and superdried X-ray-amorphous silicates.

An optionally used fine-crystalline, synthetic zeolite containing bound water is preferably zeolite A and/or P. As a zeolite of the P type, zeolite MAP (e.g. the commercial product Doucil A24 from the Crosfield company) is especially preferred. Also suitable however are zeolite X and mixtures of A, X and/or P. For example, a cocrystallization product of zeolite X and zeolite A (ca. 80 weight % of zeolite X), which is marketed by the company CONDEA Augusta S.p.A. under the brand name VEGOBOND AX® and can be described by the formula:
nNa₂O.(1−n)K₂O.Al₂O₃.(2-2.5)SiO₂.(3.5-5.5) H₂O
is also commercially available and preferably usable in the context of the present invention. Suitable zeolites display a mean particle size of less than 10 μm (volume distribution: measurement method—Coulter counter) and preferably contain 18 to 22 weight %, in particular 20 to 22 weight % of bound water.

Of course, the use of the generally known phosphates as builder substances in detergents is also possible, provided that such use has not to be avoided for ecological reasons. Particularly suitable are the sodium salts of the orthophosphates, the pyrophosphates and in particular the tripolyphosphates.

Usable organic builder substances are for example the polycarboxylic acids obtainable in the form of their sodium salts, polycarboxylic acids being understood to mean carboxylic acids which bear more than one acid function. Examples of these are citric acid, adipic acid, succinic acid, glutaric acid, malic acid, tartaric acid, maleic acid, fumaric acid, sugar acids, aminocarboxylic acids and nitrilotriacetic acid (NTA), provided that there is no objection to their use for ecological reasons, and mixtures thereof. Preferred salts are the salts of the polycarboxylic acids such as citric acid, adipic acid, succinic acid, glutaric acid, tartaric acid, sugar acids and mixtures thereof. The acids can also be used as such. In addition to their builder action, the acids typically also possess the property of an acidifying component and thus also serve for the establishment of a lower and milder pH value of surfactant-containing preparations according to the invention. Particularly to be mentioned in this context are citric acid, succinic acid, glutaric acid, adipic acid, gluconic acid and any mixtures thereof.

Further, polymeric polycarboxylates are also suitable as builders. These are for example the alkali metal salts of polyacrylic acid or polymethacrylic acid, for example those with a relative molecular weight of 500 to 70,000 g/mole.

In the context of the present invention, the molecular weights stated for polymeric polycarboxylates are weight average molecular weights M_{w} of the respective acid form, which were essentially determined by gel permeation chromatography (GPC), a UV detector being used, and the measurement being performed against an external polyacrylic acid standard, which owing to its structural similarity to the polymers tested gives realistic molecular weight values. These data differ markedly from the molecular weight data where poly-styrenesulfonic acids are used as the standard. The molecular weights measured against polystyrene acids are as a rule markedly higher than the molecular weights stated in the context of the present invention.

Suitable polymers are in particular polyacrylates, which preferably have a molecular weight from 2,000 to 20,000 g/mole. However, owing to their superior solubility, the short-chain polyacrylates from this group, which have molecular weights from 2,000 to 10,000 g/mole, particularly preferably from 3,000 to 5,000 g/mol may be preferred.

Also suitable are copolymeric polycarboxylates, in particular those of acrylic acid with methacrylic acid or of acrylic acid or methacrylic acid with maleic acid. Copolymers of acrylic acid with maleic acid which contain 50 to 90 weight % of acrylic acid and 50 to 10 weight % of maleic acid, have proved particularly suitable. Their relative molecular weight, based on the free acids, is generally 2,000 to 70,000 g/mole, preferably 20,000 to 50,000 g/mole, and in particular 30,000 to 40,000 g/mole.

The (co-)polymeric polycarboxylates can be used either as a powder or as an aqueous solution. The content of (co-)polymeric polycarboxylates in the detergents and cleaning agents according to the invention is preferably 0.5 to 20 weight %, in particular 3 to 10 weight

To improve the water-solubility, the polymers can also contain allylsulfonic acids, allyloxybenzenesulfonic acid and methallylsulfonic acid as monomers.

Also particularly preferred are biologically degradable polymers of more than two different monomer units, for example those containing as monomers salts of acrylic acid and maleic acid and also vinyl alcohol or vinyl alcohol derivatives or salts of acrylic acid and 2-alkylallyl-sulfonic acid and also sugar derivatives.

Further preferred copolymers are those that preferably contain as monomers acrolein and acrylic acid/acrylic acid salts or acrolein and vinyl acetate.

Also to be mentioned as further preferred builder substances are polymeric aminodicarboxylic acids, salts thereof, or precursor substances thereof. Particularly preferred are polyaspartic acids or salts and derivatives thereof, some of which in addition to cobuilder properties also have a bleach-stabilizing action.

Further suitable builder substances are polyacetals, which can be obtained by reaction of dialdehydes with polyolcarboxylic acids which have 5 to 7 carbon atoms and at least 3 hydroxy groups. Preferred polyacetals are obtained from dialdehydes such as glyoxal, glutaraldehyde, terephthaldehyde and mixtures thereof and from polyolcarboxylic acids such as gluconic acid and/or glucoheptonic acid.

Further suitable organic builder substances are dextrins, for example oligomers or polymers of carbo-hydrates, which can be obtained by partial hydrolysis of starches. The hydrolysis can be performed by normal, for example acid- or enzyme-catalyzed procedures. They are preferably hydrolysis products with mean molecular weights in the range from 400 to 500,000 g/mole. Here a polysaccharide with a dextrose equivalent (DE) in the range from 0.5 to 40, in particular from 2 to 30, is preferred, where DE is a common measure of the reducing action of a polysaccharide in comparison to dextrose, which has a DE of 100. Both maltodextrins with a DE between 3 and 20 and dry glucose syrups with a DE between 20 and 37 and also so-called yellow dextrins and white dextrins with higher molecular weights in the region of 2,000 to 30,000 g/mole, are usable. A preferred dextrin is described in the British patent application 9419091.

The oxidized derivatives of such dextrins are the reaction products thereof with oxidizing agents which are capable of oxidizing at least one alcohol function of the saccharide ring to the carboxylic acid function. Also suitable is an oxidized oligosaccharide, and a product oxidized at C₆ of the saccharide ring can be particularly advantageous.

Oxydisuccinates and other derivatives of disuccinates, preferably ethylenediamine disuccinate are other suitable cobuilders. Here ethylenediamine N,N′-disuccinate (EDDS) is preferably used in the form of the sodium or magnesium salts. Further, also preferred in this connection are glycerin disuccinates and glycerin trisuccinates. In zeolite-containing and/or silicate-containing formulations, suitable use amounts are about 3 to 15 weight

Further usable organic cobuilders are for example acetylated hydroxycarboxylic acids or salts thereof, which can optionally also be in lactone form and which contain at least 4 carbon atoms and at least one hydroxy group and a maximum of two acid groups.

A further substance class with cobuilder properties is represented by the phosphonates. These are in particular hydroxyalkane or aminoalkane phosphonates. Among the hydroalkane phosphonates, 1-hydroxyethane- 1,1-diphosphonate (HEDP) is of particular importance as a cobuilder. It is preferably used as a sodium salt, the disodium salt having a neutral, and the tetrasodium salt an alkaline (pH=9), reaction. Preferable amino-alkane phosphonates are ethylenediamine tetramethylene phosphonate (EDTMP), diethylenetriamine pentamethylene phosphonate (DTPMP) and higher homologs thereof. They are preferably used in the form of the neutral-reacting sodium salts, e.g. as the hexasodium salt of EDTMP or as the hepta- and octasodium salt of DTPMP. However as a builder from the phosphonates group, HEDP is preferably used. The aminoalkane phosphonates further have a pronounced heavy metal binding capacity. Hence it can be preferable, in particular if the surfactant-containing preparations according to the invention also contain bleaches, to use aminoalkane phosphonates, in particular DTPMP or to use mixtures of the said phosphonates.

Furthermore, all compounds which are capable of forming complexes with alkaline earth metals can be used as cobuilders.

Among the compounds serving as bleaching agents which release H₂O₂ in water, sodium perborate tetrahydrate and sodium perborate monohydrate are of particular importance. Other usable bleaching agents are for example sodium percarbonate, peroxypyrophosphates, citrate perhydrates and H₂O₂-releasing peracid salts or peracids, such as perbenzoates, peroxophthalates, diperazelaic acid, phthaloimino peracid or diper-dodecandioic acid. If detergent or bleaching preparations for mechanical dishwashing are produced, then bleaching agents from the group of organic bleaching agents can also be used. Typical organic bleaching agents are the diacyl peroxides, such as for example dibenzoyl peroxide. Other typical organic bleaching agents are the peroxy acids, the alkylperoxy acids and the arylperoxy acids in particular being mentioned as examples. Preferred representatives are (a) peroxybenzoic acid and ring-substituted derivatives thereof, such as alkylperoxybenzoic acids, but also peroxy-α-naphthoic acid and magnesium monoperphthalate, (b) the aliphatic or substituted aliphatic peroxy acids, such as peroxylauric acid, peroxystearic acid, ε-phthalimidoperoxycaproic acid [phthaloiminoperoxy-hexanoic acid (PAP)], o-carboxybenzamido-peroxycaproic acid, N-nonenylamidoperadipic acid and N-nonenylamido-persuccinates and (c) aliphatic and araliphatic peroxy-dicarboxylic acids, such as 1,12-diperoxycarboxylic acid, 1,9-diperoxyazelaic acid, diperocysebacic acid, diperoxybrassylic acid, the diperoxyphthalic acids, 2-decyldiperoxybutan-1,4-diacid and N,N-terephthaloyl-di-(6-aminopercaproic acid) can be used.

In order to obtain an improved bleach action during washing or cleaning at temperatures of 60° C. and lower, bleach activators can be incorporated into the surfactant-containing preparations. As bleach activators, compounds which under perhydrolysis conditions yield aliphatic peroxocarboxylic acids with preferably 1 to 10 C atoms, in particular 2 to 4 C atoms, and/or optionally substituted perbenzoic acid, can be used. Substances which bear O-and/or N-acyl groups with the stated number of C atoms and/or optionally substituted benzoyl groups are suitable. Multiply acylated alkylenediamines, in particular tetraacetyl-ethylenediamine (TAED), acylated triazine derivatives, in particular 1,5-diacetyl-2,4-dioxo-hexahydro-1,3,5-triazine (DADHT), acylated glycolurils, in particular tetraacetylglycoluril (TAGU), N-acyl-imides, in particular N-nonanoylsuccinimide (NOSI), acylated phenylsulfonates, in particular n-nonanoyl- or isononanoyloxybenzenesulfonate (n- or iso-NOBS), carboxylic acid anhydrides, in particular phthalic anhydride, acylated polyhydric alcohols, in particular triacetin, ethylene glycol diacetate and 2,5-diacetoxy-2,5-dihydrofuran, are preferred.

In addition to the conventional bleach activators or in their stead, so-called bleach catalysts can also be incorporated into the surfactant-containing preparations. These substances are bleach-reinforcing transition metal salts or transition metal complexes such as for example Mn, Fe, Co, Ru or Mo salen complexes or carbonyl complexes. Mn, Fe, Co, Ru, Mo, Ti, V and Cu complexes with N-containing tripod ligands such as Co, Fe, Cu and Ru ammine complexes are also usable as bleach catalysts.

Possible enzymes are those from the group of the proteases, lipases, amylases, cellulases or mixtures thereof. Particularly suitable are enzymatic active substances obtained from bacterial strains or fungi, such as Bacillus subtilis, Bacillus licheniformis and Streptomyces griseus. Preferably proteases of the subtilisin type and in particular proteases which are obtained from Bacillus lentus are used. Here, enzyme mixtures, for example of protease and amylase or protease and lipase or protease and cellulase or of cellulase and lipase or of protease, amylase and lipase or protease, lipase and cellulase, in particular however cellulase-containing mixtures, are of especial interest. Peroxidases or oxidases have also in some cases been found suitable. The enzymes can be adsorbed on carrier substances and/or embedded in coating substances in order to protect them against premature decomposition. The content of the enzymes, enzyme mixtures or enzyme granulates in the surfactant-containing preparations according to the invention can for example be about 0.1 to 5 weight %, preferably 0.1 to about 2 weight %.

A preferred group of suitable additives are the optical brighteners. Here the optical brighteners usual in detergents can be used. Examples of optical brighteners are derivatives of diaminostilbenedisulfonic acid or alkali metal salts thereof. For example, salts of 4,4′-bis(2-anilino-4-morpholino-1,3,5-triazinyl-6-amino-)stilbene-2,2′-disulfonic acid or similarly structured compounds, which instead of the morpholino group bear a diethanolamino group, a methylamino group, an anilino group or a 2-methoxyethylamino group are suitable. Further, brighteners of the substituted diphenylstyryl type can be contained in the part portions (of detergent-active preparations) of the surfactant-containing preparations according to the invention, e.g. the alkali metal salts of 4,4′-bis(2-sulfostyryl-)diphenyl, 4,4′-bis(4-chloro-3-sulfostyryl-)diphenyl or 4-(4-chlorostyryl-)4′-(2-sulfostyryl-)diphenyl. Mixtures of the aforesaid brighteners can also be used.

A further group of additives preferred according to the invention are UV protective substances. UV absorbers can be absorbed onto the treated textiles and improve the light stability of the fibers and/or the light stability of the other formula components. UV absorbers should be understood to mean organic substances (light filters) which are capable of absorbing ultraviolet rays and reemitting the absorbed energy in the form of long-wave radiation, e.g. heat. Examples of compounds which have these desired properties are the compounds active through non-radiative deactivation and derivatives of benzophenone with substituents in the 2- and/or 4-position. Further, substituted benzotriazoles, such as for example the water-soluble benzenesulfonic acid-3-(2H-benzotriazol-2-yl)-4-hydroxy-5-(methyl-propyl)-monosodium salt (Cibafast® H), acrylates phenyl-substituted in the 3-position (cinnamic acid derivatives), optionally with cyano groups in the 2-position, salicylates, organic Ni complexes and natural substances such as umbelliferone and the endogenous urocanic acid are suitable. Of particular importance are biphenyl derivatives and, above all, stilbene derivatives such as are for example described in EP 0728749 A and are commercially available from Ciba as Tinosorb® FD or Tinosorb® FR. As UV-B absorbers, mention can be made of 3-benzylidenecamphor and 3-benzylidene-norcamphor and derivatives thereof, e.g. 3-(4-methyl-benzylidene)-camphor, as described in EP 0693471 B 1, 4-aminobenzoic acid derivatives, preferably 4-(dimethylamino)benzoic acid 2-ethylhexyl ester, 4-(dimethylamino)benzoic acid 2-octyl ester and 4-(dimethylamino)benzoic acid amyl ester, esters of cinnamic acid, preferably 4-methoxycinnamic acid 2-ethylhexyl ester, 4-methoxy-cinnamic acid propyl ester, 4-methoxycinnamic acid isoamyl ester and 2-cyano-3,3-phenylcinnamic acid 2-ethylhexyl ester (Octocrylene), esters of salicylic acid, preferably salicylic acid 2-ethylhexyl ester, salicylic acid 4-isopropylbenzyl ester and salicylic acid homomenthyl ester, derivatives of benzophenone, preferably 2-hydroxy-4-methoxybenzophenone, 2-hydroxy-4-methoxy-4′-methylbenzophenone and 2,2′-dihydroxy-4-methoxy-benzophenone, esters of benzalmalonic acid, preferably 4-methoxybenzmalonic acid di-2-ethylhexyl ester, triazine derivatives such as for example 2,4,6-trianilino-(p-carbo-2′-ethyl-1′-hexyloxy)-1,3,5-triazine and octyl triazone, as described in EP0818450 A1 or dioctyl butamido triazone (Uvasorb® HEB), propane-1,3-diones such as for example 1-(4-tert-butylphenyl)-3-(4′ methoxyphenyl)propan-1,3-dione and ketotricyclo-(5.2.1.0)decane derivatives, as described in EP 0694521 B1. Also suitable are 2-phenylbenzimidazol-5-sulfonic acid and alkali metal, alkaline earth metal, ammonium, alkylammonium, alkanolammonium and glucammonium salts thereof, sulfonic acid derivatives of benzophenone, preferably 2-hydroxy-4-methoxybenzophenone-5-sulfonic acid and salts thereof, sulfonic acid derivatives of 3-benzylidenecamphor, such as for example 4-(2-oxo-3-bornylidenemethyl)benzene-sulfonic acid and 2-methyl-5-(2-oxo-3-bornylidene)sulfonic acid and salts thereof.

Typical UV-A filters are in particular derivatives of benzoylmethane, such as for example 1-(4′-tert-butyl-phenyl)-3-(4′-methoxyphenyl)propan-1,3-dione, 4-tert-butyl-4′-methoxydibenzoylmethane (Parsol 1789), 1-phenyl-3-(4′-isopropylphenyl)-propan-1,3-dione and also enamine compounds, as described in DE 19712033 A1 (BASF). The UV-A and UV-B filters can of course also be used as mixtures. In addition to the stated soluble substances, insoluble light-protective pigments, that is finely dispersed preferably nanoized metal oxides or salts, are also possible for this. Examples of suitable metal oxides are in particular zinc oxide and titanium dioxide and also oxides of iron, zirconium, silicon, manganese, aluminum and cerium and also mixtures thereof. As salts, silicates (talc), barium sulfate or zinc stearate can be used. The oxides and salts are already used in the form of the pigments for skin care and skin protection emulsions and decorative cosmetics. The particles here should have a mean diameter of less than 100 nm, preferably between 5 and 50 nm and in particular between 15 and 30 nm. They can be spherical in shape, however particles having an ellipsoidal shape or deviating in other ways from the spherical form can also be used. The pigments can also be surface-treated, i.e. hydrophobized or hydrophilized. Typical examples are coated titanium dioxides, such as for example titanium dioxide T 805 (Degussa) or Eusolex® T2000 (Merck). Possible hydrophobic coating agents here are above all silicones and specifically trialkoxyoctyl-silanes or simethicones. Preferably, micronized zinc oxide is used. Further suitable UV filters can be found in the review by P. Finkel in SÖFW Journal 122, 543 (1996).

The UV absorbers are normally used in quantities from 0.01 weight % to 5 weight %, preferably from 0.03 weight % to 1 weight %.

A further preferred group of additives according to the invention are colorants, in particular water-soluble or water-dispersible colorants. Preferred here are colorants such as are normally used in the detergent, rinsing, cleaning and textile treatment agents according to the invention to improve the visual appearance of the product. The selection of such colorants presents no difficulties to the skilled person, in particular since such normal colorants have high storage stability and insensitivity to the usual components of detergent-active preparations and to light and no marked fastness to textile fibers, so that they do not stain these. According to the invention, the colorants are added to the detergent and/or cleaning agents according to the invention in amounts of less than 0.01 weight %.

A further class of additives which according to the invention can be added to the detergent and/or cleaning agents are polymers. These polymers firstly include polymers which display cobuilder properties during the washing or cleaning or rinsing, namely for example polyacrylic acids, and also modified polyacrylic acids or corresponding copolymers. A further group of polymers are polyvinylpyrrolidone, and other graying inhibitors such as copolymers of polyvinylpyrrolidone, cellulose ethers and the like. Other preferable polymers are so-called soil repellents, such as are described below in detail.

As further additives according to the invention, the detergent and cleaning agents can also contain so-called soil repellents, namely polymers which are absorbed onto fibers, which favorably influence the washability of oils and fats from textiles and hence specifically discourage resoiling. This effect becomes especially marked if a textile which has previously been washed several times with a detergent or cleaning agent according to the invention which contains these oil and fat-dissolving components becomes soiled. The preferred oil and fat-dissolving components include for example nonionic cellulose ethers such as methyl-cellulose and methylhydroxypropylcellulose with a methoxy group content from 15 to 30 weight % and a hydroxypropoxy group content from 1 to 15 weight %, each based on the nonionic cellulose ether, and the polymers of phthalic acid and/or terephthalic acid or of derivatives thereof known from the prior art, in particular polymers of ethylene tere-phthalates and/or polyethylene glycol terephthalates or anionically and/or nonionically modified derivatives thereof. Particularly preferred among these are the sulfonated derivatives of the phthalic acid and terephthalic acid polymers.

Particularly in the case of preparations in liquid or gel form, these can also contain solvents. Examples of suitable solvents are mono or polyhydric alcohols with 1 to 4 C atoms. Preferred alcohols are ethanol, propan-1,2-diol, glycerin and any mixtures thereof. The solvents can be contained in the liquid preparations in an amount from 2 to 12 weight %, based on the finished preparation.

The said additives are added to the detergent and/or cleaning agents in amounts up to 30 weight % at most, preferably 2 to 20 weight %.

This enumeration of detergent and cleaning agent components that can be present in the detergent, rinsing or cleaning agents according to the invention is by no means definitive, but merely cites the main typical components of such agents. In particular, in the case of preparations in liquid or gel form, organic solvents can also be contained in the agents. These are preferably mono- or polyhydric alcohols with 1 to 4 C atoms. Preferred alcohols in such agents are ethanol, propane-1,2-diol, glycerin and mixtures of these alcohols. In preferred embodiments, these agents contain 2 to 12 weight % of such alcohols.

According to a particular embodiment, liquid or solid detergents are particularly preferred. Also particularly preferred are detergents and cleaning agents which are suitable for delicates or mild treatment of sensitive textiles.

In particular, textile care agents, in particular textile aftertreatment agents, preferably textile conditioners, softeners or dryer cloths which contain substances which influence the dimorphism of fungi are suitable.

Depending on the desired use purpose, other components can be used. Softener compositions for rinse bath brightener use are already described in the prior art. Normally, these compositions contain a cationic quaternary ammonium salt, which is dispersed in water, as the active substance. Depending on the content of active substance in the finished softener composition, these are described as diluted, ready-to-use products (active substance content below 7 weight %) or so-called concentrates (active substance content above 7 weight %). Because of the lower volume and the consequently simultaneously decreased packaging and transport costs, the textile softener concentrates have advantages from the ecological point of view, and have become increasingly established in the market. Because of the incorporation of cationic compounds, which have only a low water-solubility, normal softener compositions are mainly in the form of dispersions, have a milky-cloudy appearance and are not transparent. For product esthetic reasons, it can however also be desired to provide the consumer with transparent, clear softeners, which are visibly different from the known products.

As a textile-softening active substance, softeners according to the invention preferably contain cationic surfactants, which have already been described in detail above. Particularly preferably, these agents according to the invention contain so-called ester quats. While there are a large number of possible compounds from this substance class, ester quats which can be prepared by reaction of trialkanolamines with a mixture of fatty acids and dicarboxylic acids, optionally subsequent alkoxylation of the reaction product and quatemization in a manner known per se, as described in DE 195 39 846, are particularly preferably used according to the invention.

The ester quats prepared in this manner are outstandingly suitable for the preparation of portions according to the invention, which can be used as softeners. Since, depending on the choice of the trialkanolamine, the fatty acids and dicarboxylic acids and the quaternizing agent, a large number of suitable products can be prepared and used in the agents according to the invention, a description of the ester quats preferably to be used according to the invention in terms of their preparation route is more precise than the statement of general formula.

The stated components which react together to give the ester quats preferably to be used can be used in varying mutual proportions. In the context of the present invention, softeners wherein a reaction product of trialkanolamines with a mixture of fatty acids in the molar ratio 1:10 to 10:1, preferably 1:5 to 5:1, which has optionally been alkoxylated and then quaternized in a manner known per se, is contained in amounts from 2 to 60, preferably 3 to 35 and in particular 5 to 30 weight %, are preferred. The use of triethanolamine is particularly preferred here, so that further preferred softeners according to the invention contain a reaction product of triethanolamine with a mixture of fatty acids in the molar ratio 1:10 to 10:1, preferably 1:5 to 5:1, which has optionally been alkoxylated and then quaternized in a manner known per se, in amounts from 2 to 60, preferably 3 to 35 and in particular 5 to 30 weight %.

As fatty acids, all acids obtained from vegetable or animal oils and fats can be used in the reaction mixture for the preparation of the ester quat. As fatty acids in the reaction mixture, it is also entirely possible to use fatty acids which are non-solid, i.e. paste or liquid, at room temperature.

Irrespective of their physical state, the fatty acids can be saturated or singly or multiply unsaturated. Of course, it is possible to use not only “pure” fatty acids, but also the industrial fatty acid mixtures obtained by cleavage from fats and oils, however these mixtures are markedly preferable from the economic point of view.

Thus in the reaction mixtures for the preparation of the ester quats for the clear aqueous softeners according to the invention, for example individual species or mixtures of the following acids can be used: caprylic acid, pelargonic acid, capric acid, lauric acid, myristic acid, palmitic acid, stearic acid, octa-decan-12-ol acid, arachic acid, behenic acid, lignoceric acid, cerotic acid, melissic acid, 10-undecenoic acid, petroselic acid, petroselaidic acid, oleic acid, elaidic acid, ricinoleic acid, linolaidic acid, α- and β-eleostearic acid, gadoleic acid, erucic acid and brassidic acid. Of course, fatty acids with an odd number of C atoms can also be used, for example undecanoic acid, tridecanoic acid, pentadecanoic acid, heptadecanoic acid, nonadecanoic acid, heneicosanoic acid, tricosanoic acid, pentacosanoic acid and heptacosanoic acid.

In the content of the present invention, the use of fatty acids of the formula XIII in the reaction mixture for the preparation of the ester quat is preferred, so that preferred softeners contain a reaction product of trialkanolamines with a mixture of fatty acids of the formula IX,
R¹—CO—OH (IX)
wherein R1-CO— stands for an aliphatic, linear or branched acyl residue with 6 to 22 carbon atoms and 0 and/or 1, 2 or 3 double bonds and dicarboxylic acids in the molar ratio 1:10 to 10:1, preferably 1:5 to 5:1, which has optionally been alkoxylated and then quatemized in a manner known per se, in amounts from 2 to 60, preferably 3 to 35 and in particular 5 to 30 weight % in the agents.

As dicarboxylic acids which are suitable for the preparation of the ester quats to be used in the agents according to the invention, saturated or singly or multiply unsaturated α, ω-dicarboxylic acids are particularly possible. For example, the saturated species oxalic acid, malonic acid, succinic acid, glutaric acid, adipic acid, pimelic acid, suberic acid, azelaic acid, sebacic acid, undecanoic and dodecanoic acid, brassylic acid, tetra- and pentadecanoic acid, thapic acid and hepta-, octa- and nonadecanoic acid, eicosanoic and heneicosanoic acid, and also phellogenic acid, may be mentioned here. However, dicarboxylic acids which correspond to the general formula XIII are preferably used in the reaction mixture, so that agents according to the invention which contain a reaction product of trialkanolamines with a mixture of fatty acids and dicarboxylic acids of the formula X,
HO—OC—[X]—CO—OH (X)
wherein X stands for an optionally hydroxy-substituted alkylene group with 1 to 10 carbon atoms, in a molar ratio 1:10 to 10:1, preferably 1:5 to 5:1, which has been optionally alkoxylated and then quaternized in a manner known per se, in amounts from 2 to 60, preferably 3 to 35 and in particular 5 to 30 weight % in the agents, are preferred.

However, among the large number of ester quats preparable and usable according to the invention, those in which the alkanolamine is treithanolamine and the dicarboxylic acid is adipic acid have proved especially useful. Thus, in the context of the present invention, agents which contain a reaction product of triethanolamine with a mixture of fatty acids and adipic acid in the molar ratio 1:5 to 5:1, preferably 1:3 to 3:1, which has then been quaternized in a manner known per se, in amounts from 2 to 60, preferably 3 to 35 and in particular 5 to 30 weight % in the agents, are particularly preferred.

The agents according to the invention—and depending on whether they are formulated as textile detergents, detergent auxiliary agents or softeners—can also be endowed with additional features. Here for example, color-transfer inhibiting compositions, agents with “anti-graying” formulae, agents with improved ironing properties, agents with special fragrance release, agents with improved soil detachment or inhibition of resoiling, antibacterial agents, UV protective agents, color refreshing agents and the like can be formulated. Some examples are explained below:

Since textile structures, in particular those of rayon, viscose staple fiber, cotton and mixtures thereof, can tend to crease because the individual fibers are sensitive to bending, kinking, pressing and crushing transverse to the fiber direction, the agents according to the invention can contain synthetic anticrease agents. These for example include synthetic products based on fatty acids, fatty acid esters, fatty acid amides, fatty acid alkylol esters, fatty acid alkylol amides or fatty alcohols, which are mostly treated with ethylene oxide, or products based on lecithin or modified phosphate esters.

Increased wearing comfort can result from the additional use of antistatic agents which are also added to the agents according to the invention. Antistatic agents increase the surface conductivity and thus enable improved discharge of charges that have formed. External antistatic agents are as a rule substances with at least one hydrophilic molecular ligand and give a more or less hygroscopic film on the surfaces. These mostly boundary layer active antistatic agents can be subdivided into nitrogen-containing (amines, amides, quaternary ammonium compounds), phosphorus-containing (phosphate esters) and sulfur-containing (alkyl sulfonates, alkyl sulfates) antistatic agents. Lauryl- (or stearyl-)dimethylbenzyl-ammonium chlorides are suitable as antistatic agents for textiles or as additives to detergents, whereby a softening effect is also obtained.

To improve the water absorption capacity and the rewettability of the treated textiles and to facilitate ironing of the treated textiles, silicone derivatives can for example be used in the agents according to the invention. These also improve the rinse behavior of the agents according to the invention because of their foam-inhibiting properties. Examples of preferred silicone derivatives are polydialkyl- or alkylaryl-siloxanes, wherein the alkyl groups have one to five C atoms and are completely or partially fluorinated. Preferred silicones are polydimethylsiloxanes, which can optionally be derivatized and are then amino-functionalized or quatemized or have Si—OH, Si—H and/or Si—Cl linkages. The viscosities of the preferred silicones at 25° C. lie in the range between 100 and 100,000 centistokes, and the silicones can be used in amounts between 0.2 and 5 weight %, based on the total agent.

Finally, the agents according to the invention can also contain UV absorbers which are absorbed onto the treated textiles and improve the stability of the fibers to light. Examples of compounds which have these desired properties are the compounds and derivatives of benzophenone with substituents in the 2 and/or 4 position, which act by non-radiative deactivation. Also suitable are substituted benzotriazoles, acrylates phenyl-substituted in the 3 position (cinnamic acid derivatives), optionally with cyano groups in the 2 position, salicylates, organic Ni complexes and natural substances such as umbelliferone and urocanic acid, which is produced in the body.

A further object of the invention is a composition for oral, dental and/or denture hygiene, containing 0.000001 to 3 weight % of substances which influence the dimorphism of fungi. These agents preferably contain 0.00001 to 0.5 weight %, in particular 0.0001 to 0.05 weight % and quite particularly preferably 0.0001 to 0.005 weight %.

In partial prostheses or dentures, both the presentation as denture-cleaning tablets, and also as mouth-rinse or mouthwash or as toothpaste are suitable. In particular here, substances which inhibit formation of hyphae are preferred. Particularly in these compositions, the substances which influence the dimorphism of fungi are selected from diterpenes, seaweed and/or propolis extracts.

The oral, dental and/or dental prosthesis hygiene agents according to the invention can for example be in the form of mouthwash, gel, liquid tooth-cleaning lotion, stiff toothpaste, denture cleaner or prosthesis adhesive cream. For these, it is necessary to incorporate the substances used according to the invention into a suitable carrier.

As carriers, for example preparations in powder form or aqueous alcoholic solutions, which as mouthwashes can contain 0 to 15 weight % of ethanol, 1 to 1.5 weight % of aromatic oils and 0.01 to 0.5 weight % of sweeteners or, as mouthwash concentrates, 15 to 60 weight % of ethanol, 0.05 to 5 weight % of aromatic oils and 0.1 to 3 weight % of sweeteners and optionally other additives, and are diluted with water before use, can also be used. The concentration of the components here must be chosen so that it is sufficiently high that after dilution the concentrations in use are no lower than the stated lower limits.

As carriers, however, gels and more or less free-flowing pastes which are expressed from flexible plastic containers or tubes and applied onto the teeth with the aid of a toothbrush can also be used. Such products contain larger amounts of humectants and binders or consistency regulators and polishing components. In addition, aromatic oils, sweeteners and water are also contained in these preparations.

As humectants here, for example glycerin, sorbitol, xylitol, propylene glycols, polyethylene glycols or mixtures of these polyols, in particular polyethylene glycols with molecular weights from 200 to 800 (from 400 to 2000) can be used be contained. Preferably, sorbitol is contained as a humectant in an amount from 25-40 weight %.

As anti-tartar agents and as demineralization inhibitors, condensed phosphates can be contained in the form of their alkali metal salts, preferably in the form of their sodium or potassium salts. Owing to hydrolytic effects, the aqueous solutions of these phosphates have an alkaline reaction. By the addition of acid, the pH of the oral, dental and/or dental prosthesis hygiene agents according to the invention is adjusted to the preferred values of 7.5-9.

Mixtures of different condensed phosphates or else hydrated salts of the condensed phosphates can also be used. The specified amounts of from 2-12 weight % are however based on the anhydrous salts. As the condensed phosphate, a sodium or potassium polyphosphate is preferably contained in an amount of from 5-10 weight % of the composition.

One preferably contained active substance is a caries-inhibiting fluorine compound, preferably from the group of fluorides or monofluorophosphates, in an amount from 0.1-0.5 weight % of fluorine. Examples of suitable fluorine compounds are sodium monofluorophosphate (Na₂PO₃F), potassium monofluorophosphate, sodium or potassium fluoride, tin fluoride or the fluoride of an organic amino compound.

As binders and consistency regulators, for example natural and synthetic water-soluble polymers such as carrageen, tragacanth, guar, starch and non-ionogenic derivatives thereof such as for example hydroxypropyl guar, hydroxyethyl starch, cellulose ethers such as for example hydroxyethyl cellulose or methylhydroxypropyl cellulose, are used. Also agar-agar, xanthan gum, pectins, water-soluble carboxyvinyl polymers (e.g. Carbopol® types), polyvinyl alcohol, polyvinyl-pyrrolidone, higher molecular weight polyethylene glycols (molecular weight 10³ to 10⁶ D). Other substances which are suitable for viscosity control are laminar silicares such as for example montmorillonite clays, and colloidal silicic acid thickeners, e.g. aerogel silicic acid or pyrogenic silicic acids.

As polishing components, all polishing agents known for this, but preferably precipitated and gel silicic acids, aluminum hydroxide, aluminum silicate, aluminum oxide, aluminum oxide trihydrate, insoluble sodium metaphosphate, calcium pyrophosphate, calcium hydrogen phosphate, dicalcium phosphate, chalk, hydroxyapatite, hydrotalcite, talc, magnesium aluminum silicate (Veegum®), calcium sulfate, magnesium carbonate, magnesium oxide, sodium aluminum silicates, e.g. zeolite A or organic polymers, e.g. polymethacrylate, can be used. The polishing agents are preferably used in smaller amounts of e.g. 1-10 weight %.

The organoleptic properties of the tooth and/or oral hygiene products according to the invention can be improved by addition of aromatic oils and sweeteners. Possible aromatic oils are all natural and synthetic aromas usual for oral, dental and/or dental prosthesis hygiene agents. Natural aromas can be used both in the form of the ethereal oils isolated from the source plants and also of the individual components isolated from these. Preferably, they contain at least one aromatic oil from the group peppermint oil, curled mint oil, aniseed oil, caraway oil, eucalyptus oil, fennel oil, cinnamon oil, geranium oil, sage oil, thyme oil, marjoram oil, basil oil, citrus oil, wintergreen oil or one or several synthetically created components of these oils isolated therefrom. Examples of the most important components of said oils are menthol, carvone, anethole, cineole, eugenol, cinnamaldehyde, geraniol, citronellol, linalool, salvene, thymol, terpinene, terpinol, methylchavicol and methyl salicylate. Examples of further suitable aromas are menthyl acetate, vanillin, ionone, linalyl acetate, rhodinol and piperitone. As sweeteners, either natural sugars such as sucrose, maltose, lactose and fructose or synthetic sweeteners such as for example saccharin sodium salt, sodium cyclamate or aspartame are suitable.

As surfactants here, in particular alkyl and/or alkenyl (oligo)glycosides are usable. Their production and use as surface-active substances are for example known from U.S. Pat. No. 3,839,318, U.S. Pat. No. 3,707,535, U.S. Pat. No. 3,547,828, DE-A 19 43 689, DE-A 20 36 472 and DE-A 30 01 064 and also EP-A 77 167. Concerning the glycoside residue, both monoglycosides (x=1), wherein a pentose or hexose residue is bound by a glycoside linkage to a primary alcohol with 4 to 16 C atoms, and also oligomeric glycosides with an oligomerization level x of up to 10 are suitable. The oligomerization level here is a statistical mean value, which is based on a homolog distribution normal for such industrial products.

Particularly suitable as an alkyl and/or alkenyl (oligo)-glycoside is an alkyl and/or alkenyl (oligo)-glucoside of the formula RO(C₆H₁₀O)ₓ—H, wherein R is an alkyl and/or alkenyl group with 8 to 14 C atoms and x has a mean value from 1 to 4. Particularly preferred are alkyl oligoglucosides based on hardened C_{12/14} coconut alcohol with a DP from 1 to 3. The alkyl and/or alkenyl glycoside surfactant can be used very sparingly, amounts from 0.005 to 1 weight % being already sufficient.

Apart from the stated alkylglucoside surfactants, other nonionic, ampholytic and cationic surfactants can also be contained, such as for example: fatty alcohol poly-glycol ether sulfates, monoglyceride sulfates, monoglyceride ether sulfates, mono- and/or dialkyl sulfosuccinates, fatty acid isethionates, fatty acid sarcosinates, fatty acid taurides, fatty acid glutamates, ether carboxylic acids, fatty acid glucamides, alkylamido-betaines and/or protein fatty acid condensates, the latter preferably based on wheat proteins. In particular, for the solubilization of the mostly water-insoluble aromatic oils, a non-ionogenic solubilizer from the group of surface-active compounds can be necessary. Particularly suitable for this purpose are for example ethoxylated fatty acid glycerides, ethoxylated fatty acid part esters or fatty acid part esters of glycerin or sorbitan ethoxylates. Solubilizers from the group of ethoxylated fatty acids in particular include addition products of 20 to 60 moles of ethylene oxide to mono- and diglycerides of linear fatty acids with 12 to 18 C atoms or to triglycerides of hydroxy fatty acids such as hydroxystearic acid or ricinoleic acid. Further suitable solubilizers are ethoxylated fatty acid sorbitan part esters; these are preferably addition products of 20 to 60 moles of ethylene oxide to sorbitan monoesters and sorbitan diesters of fatty acids with 12 to 18 C atoms. Also suitable solubilizers are fatty acid part esters of glycerin or sorbitan ethoxylates; these are preferably mono- and diesters of C₁₂-C₁₈ fatty acids and addition products of 20 to 60 moles of ethylene oxide to 1 mole of glycerin or to 1 mole of sorbitol.

As solubilizers for optionally contained aromatic oils, the oral, dental and/or dental prosthesis hygiene agents according to the invention preferably contain addition products of 20 to 60 moles of ethylene oxide to hardened or unhardened castor oil (i.e. to hydroxy-stearic acid or ricinoleic acid triglyceride), to glycerin mono- and/or distearate or to sorbitan mono-and/or distearate.

Examples of further usual additives for the oral, dental and/or dental prosthesis hygiene products are:

pigments, e.g. titanium dioxide, and/or colorants;

pH regulators and buffer substances such as for example sodium bicarbonate, sodium citrate, sodium benzoate, citric acid, phosphoric acid or acid salts, e.g. NaH₂PO₄;

wound-healing and inflammation-inhibiting substances such as for example allantoin, urea, panthenol, azulene and camomile extract;

other substances active against dental tartar such as for example organophosphonates, e.g. hydroxyethane-diphosphonate or azacycloheptanediphosphonate;

preservatives such as for example sorbic acid salts or p-hydroxybenzoic acid esters; and

plaque inhibitors such as for example hexachloro-phene, chlorhexidine, hexetidine, triclosan, bromchlorophen and phenylsalicylic acid esters.

In a particular embodiment, the composition is a mouth rinse, a mouthwash, a prosthesis cleaner or a prosthesis adhesive.

These compositions are used either undiluted or as a concentrate. In addition to the usual components, the concentrates and the prosthesis cleaners thus preferably contain 0.001 to 1, in particular 0.001 to 0.5, and quite particularly preferably 0.005 to 0.1 weight %, and the mouth rinses and mouthwashes to be used undiluted preferably contain 0.0001 to 0.5, in particular 0.0001 to 0.1 and quite particularly preferably 0.0001 to 0.05 weight %, of substances which influence the dimorphism of fungi.

For prosthesis cleaners preferred according to the invention, in particular prosthesis cleaning tablets and powders, in addition to the ingredients already stated for oral, dental and/or oral prosthesis hygiene, per-compounds such as for example peroxyborate, peroxymonosulfate or percarbonate are also suitable. They have the advantage that in addition to the bleaching action they simultaneously also have a deodorant and/or disinfecting action. The incorporation of such per-compounds in prosthesis cleaners is between 0.01 and 10 weight %, in particular between 0.5 and 5 weight %.

As further components, enzymes such as for example proteases and carbohydrases, for the degradation of proteins and carbohydrates, are also suitable. The pH can lie between pH 4 and pH 12, in particular between pH 5 and pH 11.

For the prosthesis cleaning tablets, still further additives are also necessary, such as for example agents which have an effervescent effect, e.g. CO₂-releasing substances such as sodium hydrogen carbonate, fillers, e.g. sodium sulfate or dextrose, lubricants, e.g. magnesium stearate, flow regulators such as for example colloidal silicon dioxide and granulating agents such as the aforementioned high molecular weight polyethylene glycols or polyvinylpyrrolidone.

Prosthesis adhesives can be provided as powders, creams, films or liquids and boost the adhesion of the prostheses.

Natural and synthetic swelling agents are suitable. As well as alginates, plant gums, such as for example gum arabic, tragacanth and crystal gum and also natural rubber can be regarded as natural swelling agents. In particular, alginates and synthetic swelling agents, such as for example sodium carboxymethylcellulose, high molecular weight ethylene oxide copolymers, salts of poly(vinyl-ether-co-maleic acid) and polyacrylamide have themselves.

Particularly suitable as additives for paste and liquid products are hydrophobic bases, in particular hydrocarbons, such as for example white vaseline (DAB) or paraffin oil.

The examples presented below are intended to illustrate the invention further, without restricting it to them.

### EXAMPLES

1. The Adhesion Test on Textiles

For assessment of the inhibitory action of different substances on the adhesion of fungi, a procedure with which this adhesion to textiles can be determined semi-quantitatively was developed.

A50 ml C. albicans culture (strain SC5314) was grown overnight with shaking in a conical flask at 30° C. up to an optical density (OD) of ca. 1 (measured at 600 nm with a spectrophotometer). After washing once in water, the cells were again incubated for one hour at 30° C. with shaking. Next, 2.5 ml of the culture were diluted in 22.5 ml of YPD medium (1% yeast extract, 2% peptone, 2% glucose) or YPS medium (1% yeast extract, 2% peptone, 10% horse serum). The YPD cultures were incubated with shaking for 2 hours at 30° C., and the YPS cultures for 2 hours at 37° C. for the induction of hyphae.

As desired, test substance was added to the media. In addition a 2.5×2.5 cm piece of test fabric (polyester microfibers) was added to the media. After incubation, the textile pieces were washed three times with wash liquor (Perwoll powder, 12 ml/I water) and twice with water (5 min. with shaking each time), then transferred to a 6-well plate and covered with a layer of YPD-tetrazolium blue agar (1% yeast extract, 2% peptone, 2% glucose, 1.5% agar, 0.5 mg/ml tetrazolium blue chloride).

The depth of the blue coloration due to enzymatic conversion of the dyestuff tetrazolium blue chloride (3,3′-[3,3′-dimethoxy(1,1′-biphenyl)-4,4′-diyl]-bis-[2,5-diphenyl-2H-tetrazolium] dichloride (Sigma, T4375) by cellular dehydrogenases of adhered C. albicans served as a measure for the number of adhered Candida cells and was measured by measurement of the relative light reflection (measurement geometry d/8°) with a differential colorimeter (Dr. Lange Micro Color). The instrument was calibrated in accordance with the manufacturer's instructions. For this, the textile patches were dissolved out from the agar and subjected to the measurement with the differential colorimeter. In accordance with DIN 5033, the differential colorimeter determines the diffuse reflection of the samples at an angle of 8°.

2. The Growth Test

The growth rates were measured over 10 hours in the YPD medium already described above. The optical density was initially set at 0.1 OD and the cultures were incubated at 30° C.

Here it was assumed that an optical density of 1 measured at wavelength 600 nm corresponds to about 10⁷ cells.

3. The Test for Inhibition of the Formation of Hyphae

An initial culture of the Candida albicans strain SC5314 was grown overnight in minimal medium (0.67% yeast nitrogen base w/o amino acids (Difco), 2% glucose) up to an optical density of 1 at 600 nm, and washed once in sterile double-distilled H₂O. For the induction of hyphae, 100 μl to 900 μl of YPS (1% yeast extract, 2% peptone, 10% horse serum (Sigma)) were added, and incubated at 37° C. with shaking. To test the inhibitory action of different substances, farnesol (50 μl/l), propolis extract (200 ppm in ethanolic solution, Sigma, P 5182, origin: Alsace, France) or fucoidan (2 mg/ml, Sigma, F 5631) were added to the medium. A culture of 100 μl of cell suspension in 900 μl of YPD medium (1% yeast extract, 2% peptone, 2% glucose) which was incubated at 30° C. with shaking was used as the control. To quantify the formation of hyphae, 2×100 cells were counted under the microscope (Zeiss Axioscope) every 20 min. and the germ tubes formed calculated as a percentage of the total cells.

Results

TABLE 1

| | Concentration | Relative light | Growth rate |
|---|---|---|---|
| Test Substance | (wt. %) | reflection | (hr⁻¹) |
| Reference (YPD) | — | 48.7 | 0.55 |
| Farnesol | 0.0025 | 49.3 | 0.51 |
| Propolis extract | 0.002 | 48.7 | 0.37 |
| (in ethanol) | | | |
| Ethanol | 2 | not | 0.5 |
| | | determined | |
| Test (YPS) | — | 44.7 | — |

TABLE 2
Hyphae formation test results

| | Number of hyphal cells formed | | | | |
|---|---|---|---|---|---|
| | YPD | | YPS + farnesol | YPS + propolis | YPS + fucoidan |
| t (min.) | (reference) | YPS (hyphae) | (0.005 wt. %) | (0.002 wt. %) | (0.2 wt. %) |
| 0 | 0 | 0 | 0 | 0 | 0 |
| 20 | 0 | 0 | 0 | 0 | 0 |
| 40 | 0 | 15 | 0 | 0 | 4 |
| 60 | 0 | 75 | 3 | 2 | 25 |

TABLE 3
Hyphae formation inhibiting concentration of other substances

| | Effective inhibitory |
|---|---|
| Substance | concentration (wt. %) |
| Cedar leaf oil | 0.10% |
| Aniseed oil | 0.10% |
| Anethole | 0.10% |
| Geraniol | 0.01% |
| Trans-cinnamic acid | 0.01% |
| Cinnamon oil | 0.01% |
| Citronella oil | 0.001% |

It was found that under the chosen conditions only hyphae (in the YPS cultures) were capable of adhering to textile fabric. Yeast cells (YPD culture) were washed out completely, and no blue coloration of the fabric was seen. The adhered hyphae (YPS) caused a reduction in the relative light reflection compared to yeast cells (YPD, reference), while the light reflection for the cells induced in the presence of the test substances did not deviate significantly from the control value.

Both farnesol and also the propolis extract showed a reduction in the adhesion of fungi to textiles down to almost complete inhibition of the adhesion.

At the same time, as can be seen from the growth rates stated in Table 1, the test substances showed no or hardly any fungicidal or fungistatic properties at the concentrations used, but essentially complete prevention (farnesol and propolis) or marked reduction (fucoidan) in hyphae formation.

4. Wash Tests: Contamination with Candida albicans

The wash tests were performed in standard domestic washing machines (Miele W 918 Novotronic), which had previously been disinfected, in the delicates program at 30° C. The fungal cells were applied onto textile supports (polyamid, 2.5×2.5 cm) (2×10⁵ cells/microbe support) and washed together with 3.5 kg of disinfected fill laundry with the detergent formulae. As the control, the wash was performed with the same detergent without farnesol. After the washing and drying in the air, both the residual microbial burden and also the microbial transfer to other, sterile textiles were determined.

Results

The experiments were performed with the detergents A (addition of farnesol) and B (reference).

TABLE 4
Liquid detergent with farnesol

Amount in weight %

| Raw material | Detergent A | Detergent B |
|---|---|---|
| C₁₂-C₁₈ fatty alcohol + 7 EO | 15 | 15 |
| (Dehydol LT 7, Cognis) | | |
| C₁₂-C₁₄ fatty alcohol + 2 EO- | 7 | 7 |
| sulfate, sodium salt (Texapon | | |
| N 70, Cognis) | | |
| C₈₋₁₈ fatty acid cut | 8 | 8 |
| (coconut oil fatty acid, | | |
| Edenor K 12-18, Cognis) | | |
| Sodium citrate | 1.5 | 1.5 |
| Enzymes | + | + |
| Colorant | + | + |
| Perfume | + | + |
| Farnesol | 0.4 | — |
| Water | to 100 | to 100 |

75 ml of the liquid detergent is added; this corresponds to a farnesol concentration of 20 ppm in the wash liquor.

TABLE 5
Residual microbial burden

| | Residual |
|---|---|
| | Candida albicans |
| | burden Colony- |
| | forming units |
| | microbe support |
| | (CFU/MS) |
| Detergent A | 90 |
| Detergent B | 650 |
| (reference) | |

TABLE 6
Cross contamination

| | Cross contamination with |
|---|---|
| | Candida albicans (CFU/MS) |
| Detergent A | 0 |
| Detergent B (reference) | 5.2 |

Through the addition of farnesol, the residual microbial burden (see Table 5) could be drastically reduced and cross contamination of other laundry items was completely prevented (Table 6).

5. Predispensed Liquid Detergent in Polyvinyl Alcohol Film With Farnesol

TABLE 7

| | Amount in weight % | |
|---|---|---|
| Raw material | Detergent C | Detergent D |
| C₁₂-C₁₄ fatty alcohol + 5 EO + 4 PO | 25 | 25 |
| (Marlox MO 154, Sasol) | | |
| Dodecylbenzenesulfonate | 24.5 | 24.5 |
| isopropylammonium salt | | |
| (LAS-MIPA, Sasol) | | |
| C₈₋₁₈ fatty acid cut | 17.5 | 17.5 |
| (coconut oil fatty acid, Edenor K 12-18, | | |
| Cognis) | | |
| Ethanol | 3.5 | 3.5 |
| Sodium citrate | 0.6 | 0.6 |
| Enzymes | 2.0 | 2.0 |
| Water | 6.0 | 6.0 |
| Farnesol | 0.6 | — |
| Colorant | + | + |
| Perfume | + | + |
| Propylene glycol | to 100 | to 100 |

50 ml of the predispensed detergent is added; this corresponds to a farnesol concentration of 20 ppm in the wash liquor.

6. Powder Detergent with Farnesol

TABLE 8

Amount in weight %

| Raw material | Detergent E | Detergent F |
|---|---|---|
| C₁₀-C₁₃ alkylbenzenesulfonate | 13.4 | 13.3 |
| C₁₂-C₁₈ alkyl sulfate | 5.6 | 5.5 |
| C₁₂-C₁₈ alcohol with 7 EO | 5.4 | 5.3 |
| C₁₂-C₁₈ alcohol with 4.5 EO | 0.6 | 0.6 |
| Soil repellent | 0.7 | 0.7 |
| C₁₆-C₁₈ fatty acid | 0.8 | 0.8 |
| (Edenor ST1 C₁₆-C₁₈, Cognis) | | |
| Polyethylene glycol | 1.8 | 1.8 |
| Molecular weight = 4000 g/mole | | |
| Phosphonate | 1.0 | 1.0 |
| Polyacrylates | 2.9 | 2.8 |
| Carboxymethylcellulose | 0.9 | 0.9 |
| Polyvinylpyrrolidone | 0.5 | 0.5 |
| Zeolite (anhydrous active | 32.2 | 32.1 |
| substance) | | |
| Sodium carbonate | 4.5 | 4.5 |
| Sodium tricitrate | 3.7 | 3.6 |
| Citric acid | 3.7 | 3.7 |
| Sodium hydrogen carbonate | 4.9 | 4.9 |
| Sodium sulfate | 3.9 | 3.8 |
| Antifoaming agent | + | + |
| Enzymes | + | + |
| Colorant | + | + |
| Perfume | + | + |
| Farnesol | — | 0.4 |
| Water/salts | to 100 | to 100 |

75 g of detergent is added; this corresponds to a farnesol concentration of 20 ppm in the wash liquor.

Farnesol can also be incorporated as a component of the perfume. It is then contained in the perfume oil in concentrations of 0. 1 to 80% and is introduced into the wash liquor via the perfume oil contained in the detergent formula.

7. Mouthwash

TABLE 9

| | Weight % |
|---|---|
| Ethanol (96%) | 65 |
| Polyoxyethylene sorbitan | 2.0 |
| monolaureate | |
| (Tween ® 20, Uniqema) | |
| Aromatic oil | 10.0 |
| Propylene glycol | 15.0 |
| Triethanolamine isostearate | 2.0 |
| Sodium saccharinate | 0.5 |
| Farnesol | 0.01 |
| Water | to 100 |

8. Tooth Cream

TABLE 10

| | Weight % |
|---|---|
| Dicalcium phosphate | 47.5 |
| Glycerin 86% DAB | 30 |
| Toothpaste aromatic oil | 1.0 |
| Carboxymethylcellulose, sodium salt | 1.2 |
| Sodium laurylsulfate | 1.0 |
| Saccharin solution 1% | 0.5 |
| Famesol | 0.02 |
| Water | to 100 |

9. Prosthesis Cleaning Agent, Powder

TABLE 11

| | Weight % |
|---|---|
| Sodium perborate monohydrate | 25 |
| Sodium sesquicarbonate | 25 |
| Trisodium phosphate anhydrous | 40 |
| Sodium laurylsulfate | 0.2 |
| Silicic acid | 0.5 |
| Aroma substances | 0.05 |
| Farnesol | 0.5 |
| Maltodextrin | 9.3 |

10. Denture Adhesive

TABLE 12

| | Weight % |
|---|---|
| Sodium alginate | 10 |
| Paraffin oil perliquidum | 90 |
| Farnesol | 0.01 |

1. A method of inhibiting the formation of hyphae in fungi comprising: contacting said fungi with a composition comprising propolis extracts, plant extracts, cinnamic acid, terpenes, propolis extracts derivatives, plant extracts derivatives, cinnamic acid derivatives, terpene derivatives, or combinations thereof.

2. The method of claim 1 wherein said terpene or terpene derivative is monoterpene, sesquiterpene, diterpene, monoterpene derivatives, sesquiterpene derivatives, or diterpene derivatives.

3. The method of claim 1 wherein said composition comprises seaweed extracts, fucoidin, anethole, geraniol, farnesol or farnesoic acid.

4. The method of claim 1 wherein said composition has a non-fungicidal or non-fungistatic final concentration.

5. The method of claim 1 wherein said fungi are Candida.

6. The method of claim 1 wherein said fungi are C. aaseri, C. actis-condensi, C. acutus, C. agrestis, C. albicans, C. amapae, C. anatomiae, C. ancudensis, C. antarctica, C. antillancae, C. apicola, C. apis, C. aquaetextoris, C. aquatica, C. atlantica, C. atmosphaerica, C. auringiensis, C. azyma, C. beechii, C. benhamii, C. bertae, C. berthetii, C. blankii, C. boidinii, C. boleticola, C. bombi, C. bondarzewiae, C. brumptii, C. buffonii, C. buinensis, C. cacaoi, C. canterellii, C. capsuligena, C. cariosilignicola, C. caseinolytica, C. castellii, C. catenulata, C. chalmersi, C. chilensis, C. chiropterorum, C. ciferii, C. claussenii, C. coipomensis, C. colliculosa, C. conglobata, C. curiosa, C. cylindracea, C. dendrica, C. dendronema, C. deserticola, C. diddensiae, C. diffluens, C. diversa, C. drymisii, C. dubliniensis, C. edax, C. entomophila, C. eremophila, C. ergatensis, C. ernobii, C. etchellsii, C. etchellsii, C. ethanolica, C. ethanothermophilum, C. evantina, C. fabianii, C. famata, C. fennica, C. flareri, C. fluviotilis, C. fragariorum, C. fragi, C. fragicola, C. freyschussii, C. friedrichii, C. fructus, C. fusiformata, C. geochares, C. glabrata, C. glaebosa, C. graminis, C. gropengiesseri, C. guilliermondii, C. haemulonii, C. hellenica, C. heveanensis, C. holmii, C. homilentoma, C. humicola, C. humilis, C. insectorum, C. intermedia, C. ishiwadae, C. japonica, C javanica, C. karawaiewii, C. kefyr, C. kruisii, C. krusei, C. krusoides, C. lactiscondensi, C. lambica, C. laureliae, C. lipolytica, C. llanquihuensis, C. lodderae, C. lusitaniae, C. magnoliae, C. malicola, C. maltosa, C. maris, C. maritima, C. melibiosica, C. melinii, C. membranaefaciens, C. mesenterica, C. methanosorbosa, C. milleri, C. mogii, C. molischiana, C. monosa, C. montana, C. mucilaginosa, C. multisgemmis, C. musae, C. muscorum, C. mycoderma, C. naeodendra, C. nakasei, C. nemodendra, C. nitratophila, C. norvegensis, C. novakii, C. oleophila, C. oregonensis, C. palmyrana, C. paludigena, C. parapsilosis, C. pararugosa, C. pelliculosa, C. peltata, C. periphelosum, C. petro-huensis, C. pignaliae, C. pintolopesii, C. pinus, C. placentae, C. polymorpha, C. populi, C. pseudo-tropicalis, C. psychrophila, C. pulcherrima, C. punica, C. quercitrusa, C. quercuum, C. railenensis, C. ralunensis, C. reukaufli, C. rhagii, C. rugo-pelliculosa, C. rugosa, C. saitoana, C. sake, C. salmanticensis, C. santamariae, C. santjacobensis, C. savonica, C. schatavii, C. sequanensis, C. shehatae, C. silvae, C. silvanorum, C. silvicultrix, C. solani, C. sonorensis, C. sophiae-reginae, C. sorboxylosa, C. spandovensis, C. sphaerica, C. stellata, C. stellatoidea, C. succiphila, C. sydowiorum, C. tanzawaensis, C. tenuis, C. tepae, C. terebra, C. torresii, C. tropicalis, C. tsuchiyae, C. tsukubaensis, C. utilis, C. valdiviana, C. valida, C. vanderwaltii, C. vartiovaarai, C. versatilis, C. vini, C. viswanathii, C. wickerhamii, C. xestobii or C. zeylanoides.

7. The method of claim 6 wherein said fungi are Candida albicans or Candida glabrata.

8. The method of claim 1 wherein said fungi are present on a surface.

9. The method of claim 8 wherein said surface is the human body, textiles, ceramics, plastics, laundry, prostheses or dentures.

10. The method of claim 1 wherein said propolis extracts, plant extracts, cinnamic acid, terpenes, propolis extracts derivatives, plant extracts derivatives, cinnamic acid derivatives, terpene derivatives, or combinations thereof are present at a final concentration of about 0.000001% by weight to about 3% by weight.

11. The method of claim 1 wherein said propolis extracts, plant extracts, cinnamic acid, terpenes, propolis extracts derivatives, plant extracts derivatives, cinnamic acid derivatives, terpene derivatives, or combinations thereof are present at a final concentration of about 0.0001% by weight to about 0.1% by weight.

12. A composition for inhibiting the formation of hyphae in fungi, comprising:
propolis extracts, plant extracts, cinnamic acid, terpenes, propolis extracts derivatives, plant extracts derivatives, cinnamic acid derivatives, terpene derivatives, or combinations thereof.

13. The composition of claim 12 further comprising a detergent, cleaning agent, oral hygiene agent, dental hygiene agent, or textile treatment agent.

14. The composition of claim 12 wherein said propolis extracts, plant extracts, cinnamic acid, terpenes, propolis extracts derivatives, plant extracts derivatives, cinnamic acid derivatives, terpene derivatives, or combinations thereof are present at a final concentration of about 0.000001% to about 3% by weight.

15. A textile treatment agent comprising the composition of claim 12.

16. The textile treatment agent of claim 15 wherein said agent is textile conditioners, softeners, or dryer cloths.

17. An oral hygiene agent or dental hygiene agent comprising the composition of claim 12.

18. The oral hygiene agent or dental hygiene agent of claim 17, wherein said oral hygiene agent or dental hygiene agent is a mouth rinse, a mouthwash, a prosthesis cleaner or a prosthesis adhesive.
